(19) United States
(12) Patent Application Publication
(10) Pub. No.: US 2025/0354107 A1
(43) Pub. Date: Nov. 20, 2025

(54) Device and control method for observing biofilm formation mechanism by multi-sequence pressure-holding sampling

(71) Applicant: Guangdong University of Technology, Guangzhou (CN)

(72) Inventors: Jingchun FENG, Guangzhou (CN); Si ZHANG, Guangzhou (CN); Song ZHONG, Guangzhou (CN); Yi WANG, Guangzhou (CN); Zhifeng YANG, Guangzhou (CN)

(73) Assignee: Guangdong University of Technology, Guangzhou (CN)

(21) Appl. No.: 18/663,127

(22) Filed: May 14, 2024

(51) Int. Cl. C12M 1/34; C12M 1/00; C12M 1/36

(52) U.S. Cl. CPC C12M 41/36; C12M 29/04; C12M 41/48

(57) ABSTRACT

A device for observing a biofilm formation mechanism by multi-sequence pressure-holding sampling, including a culture kettle and a film formation and degradation unit is provided. The top of the culture kettle has a viewing window and a motor. An output shaft of the motor extends into the culture kettle and is coupled with the center of the top of the film formation and degradation unit. The film formation and degradation unit includes a fixed tray and perforations distributed circumferentially on the surface of the fixed tray. An upper end surface of each perforation is removably fixed with a handle, a lower end of the handle is fixed with a film sheet exposed below the fixed tray after passing through the corresponding perforation. The top of the culture kettle also has a pressure-holding telescopic unit with the handle in the vertical direction used to grip any handle.

CROSS REFERENCE TO THE RELATED APPLICATIONS

This application claims priority to Chinese Patent Application No. 202310186004.2, filed on Mar. 1, 2023, the entire contents of which are incorporated herein by reference.

### TECHNICAL FIELD

The present invention relates to the technical field of microorganism culturing, and more particularly to a device and a control method for observing a biofilm formation mechanism by multi-sequence pressure-holding sampling.

### BACKGROUND

It is estimated that there are about 4 trillion pieces of plastic floating on the sea surface worldwide, accounting for 73% of all plastic fragments, which are evenly distributed in the sea water in the Antarctic and Arctic regions, as well as in the sediments of the Mariana Trench having a depth of nearly 10000 meters, indicating serious potential risks of plastics to the environment. Microorganisms can decompose plastics of many common materials (such as PE, PS, PET, etc.) through the action of biological enzymes. At present, a variety of microorganisms that can degrade PE plastics have been separated from the sea, including fungi (such as Aspergillus), bacteria (such as kocuria, Bacillus, Alcanivorax, Halomonas, etc.).

A biofilm, also known as a bacterial biofilm, is an organized community of bacteria that adheres to the surface of an animate or inanimate object and is wrapped by bacterial extracellular macromolecules. The complexity of the biofilm allows bacterial cells to survive in a wide range of environments and facilitates cellular dispersion to colonize new areas.

For the formation and degradation mechanism of biofilms on a plastic surface in a marine environment, existing technologies generally do research on a single colony or under normal pressure, and rarely explore the formation and degradation mechanism of the biofilms on the plastic surface under a high-pressure environment. As a result, its formation process cannot be fully reflected, bringing understanding deviation to the exploration of the formation and degradation mechanism of the biofilms on the plastic surface. In light of this, patent application document CN114350507A discloses a single colony separation device in a deep-sea in-situ environment, which can realize separation, selection and cultivation of single colonies in a pressure-holding condition, and ultimately obtain target bacteria, thereby effectively improving the culturability of marine microorganisms, and providing a basic solution for the separation and cultivation of the marine microorganisms.

However, the above device is designed to separate, select and cultivate single colonies. Although the device is further provided with a sampling device, the sampling device occupies a considerable space in an incubator because it needs to be moved precisely by means of an inner slide rail to obtain desired colonies. As a result, it is not conducive to the miniaturization of the device, and the reliability is low. Moreover, the device is not capable of sampling in a liquid culture environment, which further limits the exploration of the biofilm formation mechanism.

### SUMMARY

In view of the above problems, the present invention provides a device and a control method for observing a biofilm formation mechanism by multi-sequence pressure-holding sampling, which mainly solves the problems that an existing high-pressure microorganism sampling device occupies a considerable space in an incubator and is difficult to grip a film sheet in a liquid environment.

To solve the above technical problems, a first aspect of the present invention provides a device for observing a biofilm formation mechanism by multi-sequence pressure-holding sampling, including: a culture kettle, and a film formation and degradation unit disposed inside the culture kettle, wherein the top of the culture kettle is provided with a viewing window and a motor, an output shaft of the motor extends into the culture kettle and is coupled with the center of the top end of the film formation and degradation unit, the film formation and degradation unit includes a fixed tray and perforations distributed circumferentially on the surface of the fixed tray, an upper end surface of each perforation is removably fixed with a handle, a lower end of the handle is fixed with a film sheet which is exposed under the fixed tray after passing through the corresponding perforation, the top of the culture kettle is also provided with a pressure-holding telescopic unit coaxial with the handles, and a movable end of the pressure-holding telescopic unit is used to grip the handles.

In some embodiments, the pressure-holding telescopic unit includes a cylinder fixed at the top of the culture kettle, the top of the cylinder is provided with a first valve, a lower side wall of the cylinder is provided with a pneumatic valve and a second valve, the second valve is located between the first valve and the pneumatic valve, a piston is disposed inside the cylinder, the lower end of the piston is provided with a connecting rod, and the lower end of the connecting rod is provided with a gripping member for gripping the handle.

In some embodiments, the cylinder includes a first fixed cylinder fixed at the top of the culture kettle, and a second fixed cylinder removably mounted at the upper end of the first fixed cylinder; when the piston moves to the top of the second fixed cylinder, the horizontal height of the gripping member is greater than the horizontal height of the pneumatic valve; and when the piston moves to the bottom of the second fixed cylinder, the horizontal height of the gripping member is greater than or equal to the horizontal height of the top of the handle.

In some embodiments, the first fixed cylinder and the second fixed cylinder are removably connected by means of a movable joint.

In some embodiments, the gripping member is a magnet and the handle is machined from a magnetic metal.

In some embodiments, the device for observing a biofilm formation mechanism by multi-sequence pressure-holding sampling further includes a pressure control unit, and an output end of the pressure control unit is connected to the culture kettle and the first valve.

In some embodiments, the pressure control unit includes an air compressor, a pressure increasing valve, an air storage tank, a pressure regulating valve and an air delivery valve connected in sequence, and the air delivery valve is connected to the culture kettle and the first valve via an air delivery pipe.

In some embodiments, the top of the culture kettle is also provided with a temperature sensor, a pressure sensor, and a vent valve.

In some embodiments, the film sheet is not in contact with the bottom of the culture kettle.

A second aspect of the present invention provides a control method for observing a biofilm formation mechanism by multi-sequence pressure-holding sampling, wherein the control method is applicable to the device described above, and includes the following steps of:

In a cultivation process, activating the motor, which controls the fixed tray to rotate at a predetermined speed for a predetermined period of time; and

In a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

The present invention bas the beneficial effects that the film formation and degradation unit and the pressure-holding telescopic unit cooperate with each other in the vertical direction, so that the area capable of allowing microorganism culture is increased to the maximum extent in the limited space of the culture kettle, which is conducive to the miniaturization of the device; in addition, the reliability is high; and meanwhile, the problem that it is difficult to grip the film sheet in a pressure-holding manner in the liquid high-pressure environment is solved.

### BRIEF DESCRIPTION OF THE DRAWINGS

FIG. 1 is a schematic structural diagram of a device for observing a biofilm formation mechanism by multi-sequence pressure-holding sampling as disclosed in Embodiment 1 of the present invention;

FIG. 2 is a schematic structural diagram of a fixed tray as disclosed in Embodiment 1 of the present invention;

FIG. 3 is a schematic diagram of a mounting structure of a handle and a film sheet as disclosed in Embodiment 1 of the present invention;

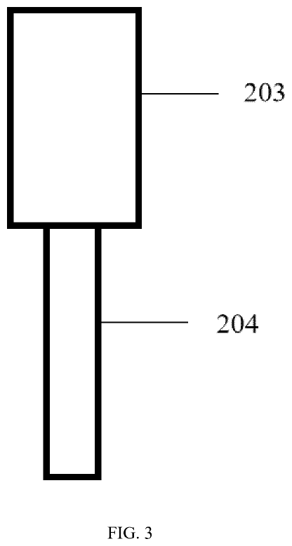

FIG. 4 is a schematic structural diagram of a pressure-holding telescopic unit as disclosed in Embodiment 1 of the present invention;

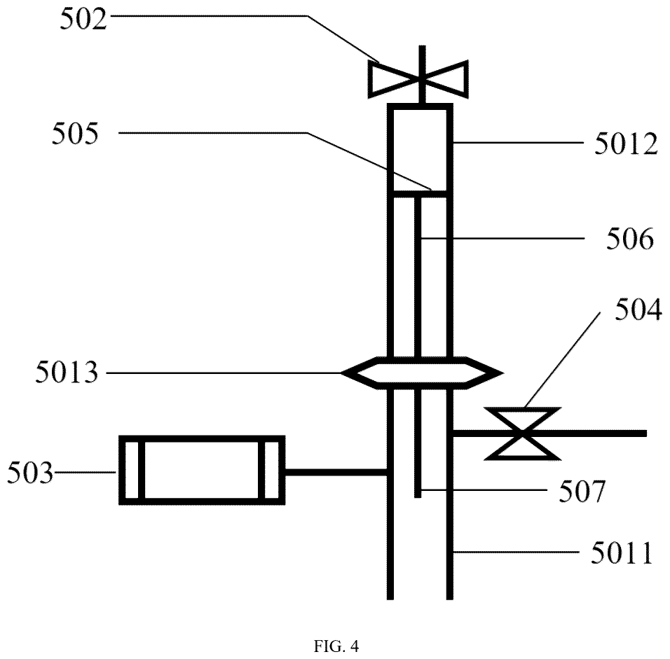

Reference numerals: 1, culture kettle; 2, film formation and degradation unit; 3, viewing window; 4, motor; 5, pressure-holding telescopic unit; 6, pressure control unit; 7, temperature sensor; 8, pressure sensor; 9, vent valve; 10, central control unit; 201, fixed tray; 202, perforation; 203, handle; 204, film sheet; 501, cylinder; 502, first valve; 503, pneumatic valve; 504, second valve; 505, piston; 506, connecting rod; 507, gripping member; 5011, first fixed cylinder; 5012, second fixed cylinder; 5013, movable joint; 601, air compressor; 602, pressure increasing valve; 603, air storage tank; 604, pressure regulating valve; 605, air delivery valve; and 606, air delivery pipe.

### DETAILED DESCRIPTION OF THE EMBODIMENTS

In order to make the objectives, technical solutions, and advantages of the present invention clearer and more definite, the content of the present invention is further explained in detail below in conjunction with the accompanying drawings and specific embodiments. It should be understood that the specific embodiments described herein are only intended to explain the present invention and are not intended to limit the present invention. Additionally, it should be noted that for ease of description, only parts related to the present invention, and not all contents, are shown in the accompanying drawings.

### Embodiment 1

The present embodiment provides a device for observing a biofilm formation mechanism by multi-sequence pressure-holding sampling, in which a film formation and degradation unit 2 cooperates with a pressure-holding telescopic unit 5 in the vertical direction, so that the area capable of allowing microorganism culture is increased to the maximum extent in the limited space of the culture kettle 1 is increased, which is conducive to the miniaturization of the device; and in addition, the reliability is high.

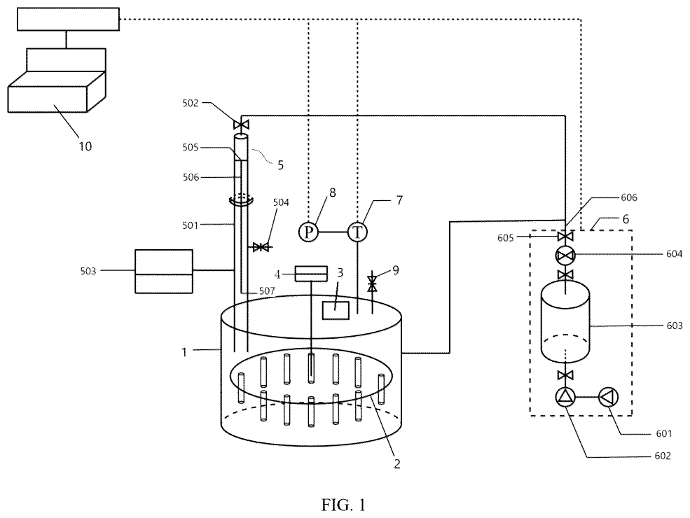

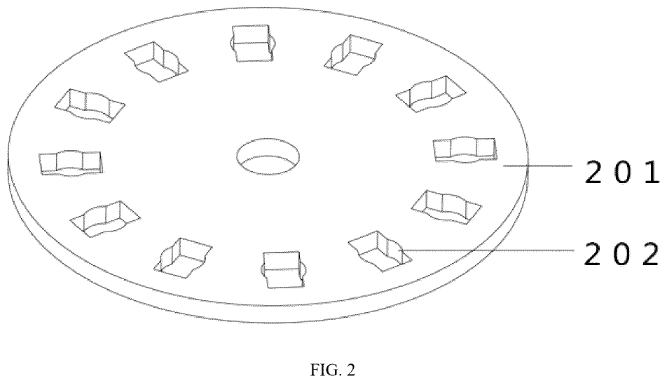

As shown in FIGS. 1 and 2, the device includes a culture kettle 1, and a film formation and degradation unit 2 disposed inside the culture kettle 1. The top of the culture kettle 1 is provided with a viewing window 3 and a motor 4, and an output shaft of the motor 4 extends into the culture kettle 1 and is coupled with the center of the top end of the film formation and degradation unit 2. The film formation and degradation unit 2 includes a fixed tray 201 and perforations 202 distributed circumferentially on the surface of the fixed tray 201. An upper end surface of each perforation 202 is removably fixed with a handle 203, the lower end of the handle 203 is fixed with a film sheet 204. The film sheet 204 is exposed under the fixed tray 201 after passing through the corresponding perforation 202. The film sheet 204 can freely pass through the corresponding perforation 202, and the handle 203 cannot freely pass through the perforations 202. The top of the culture kettle 1 is provided with a pressure-holding telescopic unit 5. The pressure-holding telescopic unit 5 is coaxial with the handles 203 in the vertical direction, and a movable end of the pressure-holding telescopic unit 5 is used to grip the handles 203.

In the present embodiment, by providing the film formation and degradation unit 2 inside the culture kettle 1, and utilizing the motor 4 to drive the film formation and degradation unit 2 to slowly rotate and stir, the material transfer in the culture kettle 1 is enhanced to increase the energy and nutrient utilization efficiency of the microorganisms, and further accelerate the reproduction efficiency of microorganisms, so that more microorganisms can adhere to the film formation and degradation device to form a biofilm and degrade the plastics. Furthermore, the top of the culture kettle 1 is also provided with a pressure-holding telescopic unit 5. The pressure of the pressure-holding telescopic unit 5 and that of the culture kettle 1 are kept the same during the sampling process, ensuring that the pressure in the culture kettle 1 is always constant to that for the in-situ state. In addition, the motor 4 is also used to drive any handle 203 on the film formation and degradation unit 2 to move directly above the pressure-holding telescopic unit 5. The handle 203 is griped by the movable end of the pressure-holding telescopic unit 5, and the pressure in the pressure-holding telescopic unit 5 is subsequently released until it reaches normal pressure; and then the film sheet 204 on the handle 203 is removed.

The above-mentioned pressure-holding telescopic unit 5 may be any device which is capable of self-pressurization and pressure relief, and meanwhile, which has a telescopic gripping function. In one example, the pressure-holding telescopic unit 5 includes a cylinder 501 fixed to the top of the culture kettle 1. The top of the cylinder 501 is provided with a first valve 502. The lower side wall of the cylinder 501 is provided with a pneumatic valve 503 and a second valve 504. The second valve 504 is located between the first valve 502 and the pneumatic valve 503. A piston 505 is disposed inside the cylinder 501. The lower end of the piston 505 is provided with a connecting rod 506. The lower end of the connecting rod 506 is provided with a gripping member 507 for gripping the handle 203. The gripping member 507 is what is called the movable end of the pressure-holding telescopic unit 5 in the context.

In the above solution, the cylinder 501, as a pressure-holding cavity, is directly communicated with the culture kettle 1. The cylinder 501 is spaced from the culture kettle 1 with the pneumatic valve 503 disposed therebetween, forming two independent pressure environments. During sampling, gas is injected to the first valve 502 to increase pressure. As the pressure increases with the injected gas, when the gas pressure of the upper part of the piston 505 is slightly larger than the pressure in the culture kettle 1, the piston 505 will move downward, and at the same time, the connecting rod 506 drives the gripping member 507 to move downward. In this process. by adjusting the rotation speed of the motor 4 and observing through the viewing window 3, the gripping member 507 can be enabled to grip one of the handles 203 on the fixed tray 201. Then, the pressure input to the first valve 502 is stopped, and by opening the first valve 502, the gas in the upper part of the piston 505 is slowly released. At this time, the piston 505 will move upward. When the gas in the upper part of the piston 505 is completely released, the pneumatic valve 503 is activated, so that the cylinder 501 and the culture kettle 1 form two independent pressure systems. Finally, the second valve 504 is opened, all the pressure of the upper part of the piston 505 is released, and the film sheet 204 located on the handle 203 can be taken out by detaching the cylinder 501. Obviously, the pressure of the upper part of the piston 505 originates from an external pressure source, and the pressure of the lower part of the piston 505 originates from the internal pressure of the culture kettle 1. In this solution, the vertical reciprocating motion of the gripping member 507 is controlled by pressure, which is high in reliability.

As one of the preferred solutions of the present invention, a structure of a detachable cylinder 501 is also provided for subsequently taking out the handle 203 and the film sheet 204 more conveniently. In this example, the cylinder 501 includes a first fixed cylinder 5011 fixed at the top of the culture kettle 1, and a second fixed cylinder 5012 detachably mounted at the upper end of the first fixed cylinder 5011. When the piston 505 moves to the top of the second fixed cylinder 5012, the horizontal height of the gripping member 507 is greater than the horizontal height of the pneumatic valve 503. When the piston 505 moves to the bottom of the second fixed cylinder 5012, the horizontal height of the gripping member 507 is slightly greater than or equal to the horizontal height of the top of the handle 203. In this solution, once the pressure relief of the second valve 504 is complete, the second fixed cylinder 5012 can be removed, then the gripping member 507 on the connecting rod 506 is taken out, and the film sheet 204 is removed. In subsequent sampling, the remaining film sheets 204 are sequentially taken out in the above way.

More preferably, the upper end of the second fixed cylinder 5012 needs to be connected to the first valve 502, which is inconvenient to be disassembled by rotating or the like. In an optional solution, the above-mentioned first fixed cylinder 5011 and the second fixed cylinder 5012 may be detachably connected by means of a movable joint 5013. In this way, the second fixed cylinder 5012 can be lifted upward only by rotating the movable joint 5013.

Optionally, the above-mentioned gripping member 507 is a magnet, and the handle 203 is machined from a magnetic metal. When the magnet is driven by the piston 505 to be in contact with the metal handle 203, the action of the magnet to absorb the handle 203 is done. Specifically, according to different experimental requirements, the film sheet 204 may be a plastic sheet of different materials, and may be connected to the lower part of the handle 203 via threads. The upper part of the handle 203 is of a groove structure, and is made of iron, etc., and the purpose is that the upper part can be absorbed by the magnet in the telescopic device. The groove matches with the magnet in shape and size.

A pressure control unit 6 is also included, and the output end of the pressure control unit 6 is connected to the culture kettle 1 and the first valve 502, respectively. In one example, the pressure control unit 6 includes an air compressor 601, a pressure increasing valve 602, an air storage tank 603, a pressure regulating valve 604 and an air delivery valve 605 connected in sequence. The air delivery valve 605 is connected to the culture kettle 1 and the first valve 502 via an air delivery pipe 606. In this solution, the pressure control unit 6 is the pressure source for the culture kettle 1 and the pressure-holding telescopic unit 5, the difference is that the pressure control unit 6 mainly provides constant culture pressure for the culture kettle 1, while for the pressure-holding telescopic unit 5, the pressure control unit 6 is used as a driving source for the pressure-holding telescopic unit 5.

The top of the culture kettle 1 is also provided with a temperature sensor 7, a pressure sensor 8 and a vent valve 9. A data end of a central control unit 10 is coupled with the temperature sensor 7, the pressure sensor 8 and the air delivery valve 605, so as to obtain the parameters fed back by the temperature sensor 7 and the pressure sensor 8, and to control the open and close of the air delivery valve 605. In the process of both culturing and sampling, the internal temperature and pressure of the culture kettle 1 can be adjusted by the parameters fed back from the temperature sensor 7 and pressure sensor 8. The temperature of the culture kettle I may be maintained at a constant temperature through heat exchange with a water bath system, while the pressure of the culture kettle 1 can be adjusted by the pressure control unit 6 and the vent valve 9. The adjustment of the temperature and pressure is a conventional technology, and will not be repeated below.

More preferably, the film sheet 204 is not in contact with the bottom of the culture kettle 1. The fixed tray 201 is suspended in the middle part of the culture kettle 1 by the output shaft of the motor 4, and the film sheet 204 hangs down naturally, maintaining a maximum area for microorganism culturing.

### Embodiment 2

The present embodiment provides a control method for observing a biofilm formation mechanism by multi-sequence pressure-holding sampling. The control method is applicable to the device described in Embodiment 1, and includes the following steps of:

in a cultivation process, activating the motor 4, which controls the fixed tray 201 to rotate at a predetermined speed for a predetermined period of time; and

in a sampling process, driving, by the motor 4, the fixed tray 201 to rotate to enable the handle 203 as a target to be located directly under the pressure-holding telescopic unit 5, activating the pressure-holding telescopic unit 5 to increase pressure, when the internal pressure of the pressure-holding telescopic unit 5 is consistent with the internal pressure of the culture kettle 1, controlling the movable end of the pressure-holding telescopic unit 5 to go down to enter into the culture kettle 1 to grip the handle 203 as the target, then, carrying the handle 203 as the target, by the movable end of the pressure-holding telescopic unit 5, to return to its original position, disconnecting a connecting channel between the pressure-holding telescopic unit 5 and the culture kettle 1, relieving pressure by the pressure-holding telescopic unit 5 until the internal pressure of the pressure-holding telescopic unit 5 reaches normal pressure, taking out the handle 203 as the target from the interior of the pressure-holding telescopic unit 5, and removing the film sheet 204 carried from the handle 203 as the target.

The telescopic motion of the movable end of the pressure-holding telescopic unit 5 is controlled by the first valve 502. Specifically, the first valve 502 on the second fixed cylinder 5012 is opened to relieve the pressure, so that the gripping member 507 carrying the handle 203 as the target returns to its original position.

The connecting channel between the pressure-holding telescopic unit 5 and the culture kettle I is controlled by means of the pneumatic valve 503. Specifically, the air pressure communication between the pressure-holding telescopic unit 5 and the culture kettle 1 is disconnected by closing the pneumatic valve 503.

The above embodiments are only intended to illustrate the technical concept and characteristics of the present invention, and their purpose is to enable those of ordinary skills in the art to understand the content of the present invention and implement it accordingly, and not to limit the scope of the present invention. All equivalent variations or modifications made according to the essence of the content of the present invention should be covered by the scope of the present invention.

What is claimed is:

1. A device for observing a biofilm formation mechanism by multi-sequence pressure-holding sampling, comprising:
a culture kettle, and
a film formation and degradation unit disposed inside the culture kettle, wherein
the top of the culture kettle is provided with a viewing window and a motor, an output shaft of the motor extends into the culture kettle and is coupled with the center of the top end of the film formation and degradation unit,
the film formation and degradation unit comprises a fixed tray and perforations distributed circumferentially on the surface of the fixed tray, an upper end surface of each perforation is removably fixed with a handle, a lower end of the handle is fixed with a film sheet, the film sheet is exposed below the fixed tray after passing through the corresponding perforation, the top of the culture kettle is also provided with a pressure-holding telescopic unit coaxial with the handles in the vertical direction, and a movable end of the pressure-holding telescopic unit is used to grip the handles.

2. The device for observing a biofilm formation mechanism by multi-sequence pressure-holding sampling according to claim 1, wherein
the pressure-holding telescopic unit comprises a cylinder fixed at the top of the culture kettle, the top of the cylinder is provided with a first valve, a lower side wall of the cylinder is provided with a pneumatic valve and a second valve, the second valve is located between the first valve and the pneumatic valve, a piston is disposed inside the cylinder, the lower end of the piston is provided with a connecting rod, and the lower end of the connecting rod is provided with a gripping member for gripping the handle.

3. The device for observing a biofilm formation mechanism by multi-sequence pressure-holding sampling according to claim 2, wherein
the cylinder comprises a first fixed cylinder fixed at the top of the culture kettle, and a second fixed cylinder removably mounted at the upper end of the first fixed cylinder; when the piston moves to the top of the second fixed cylinder, the horizontal height of the gripping member is greater than the horizontal height of the pneumatic valve; and when the piston moves to the bottom of the second fixed cylinder, the horizontal height of the gripping member is greater than or equal to the horizontal height of the top end of the handle.

4. The device for observing a biofilm formation mechanism by multi-sequence pressure-holding sampling according to claim 3, wherein
the first fixed cylinder and the second fixed cylinder are removably connected by means of a movable joint.

5. The device for observing a biofilm formation mechanism by multi-sequence pressure-holding sampling according to claim 2, wherein
the gripping member is a magnet and the handle is machined from a magnetic metal.

6. The device for observing a biofilm formation mechanism by multi-sequence pressure-holding sampling according to claim 2, further comprising:
a pressure control unit, wherein the output end of the pressure control unit is connected to the culture kettle and the first valve.

7. The device for observing a biofilm formation mechanism by multi-sequence pressure-holding sampling according to claim 6, wherein
the pressure control unit comprises an air compressor, a pressure increasing valve, an air storage tank, a pressure regulating valve and an air delivery valve connected in sequence; and the air delivery valve is connected to the culture kettle and the first valve via an air delivery pipe.

8. The device for observing a biofilm formation mechanism by multi-sequence pressure-holding sampling according to claim 1, wherein the top of the culture kettle is also provided with a temperature sensor, a pressure sensor, and a vent valve.

9. The device for observing a biofilm formation mechanism by multi-sequence pressure-holding sampling according to claim 1, wherein
the film sheet is not in contact with the bottom of the culture kettle.

10. A control method for observing a biofilm formation mechanism by multi-sequence pressure-holding sampling using the device for observing a biofilm formation mechanism by multi-sequence pressure-holding sampling according to claim 1, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

11. A control method for observing a biofilm formation mechanism by multi-sequence pressure-holding sampling using the device for observing a biofilm formation mechanism by multi-sequence pressure-holding sampling according to claim 2, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

12. A control method for observing a biofilm formation mechanism by multi-sequence pressure-holding sampling using the device for observing a biofilm formation mechanism by multi-sequence pressure-holding sampling according to claim 3, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

13. A control method for observing a biofilm formation mechanism by multi-sequence pressure-holding sampling using the device for observing a biofilm formation mechanism by multi-sequence pressure-holding sampling according to claim 4, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

14. A control method for observing a biofilm formation mechanism by multi-sequence pressure-holding sampling using the device for observing a biofilm formation mechanism by multi-sequence pressure-holding sampling according to claim 5, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

15. A control method for observing a biofilm formation mechanism by multi-sequence pressure-holding sampling using the device for observing a biofilm formation mechanism by multi-sequence pressure-holding sampling according to claim 6, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

16. A control method for observing a biofilm formation mechanism by multi-sequence pressure-holding sampling using the device for observing a biofilm formation mechanism by multi-sequence pressure-holding sampling according to claim 7, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

17. A control method for observing a biofilm formation mechanism by multi-sequence pressure-holding sampling using the device for observing a biofilm formation mechanism by multi-sequence pressure-holding sampling according to claim 8, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.

18. A control method for observing a biofilm formation mechanism by multi-sequence pressure-holding sampling using the device for observing a biofilm formation mechanism by multi-sequence pressure-holding sampling according to claim 9, comprising the following steps:
in a cultivation process, activating the motor, which controls the fixed tray to rotate at a predetermined speed for a predetermined period of time; and
in a sampling process, driving, by the motor, the fixed tray to rotate to enable the handle as a target to be located directly under the pressure-holding telescopic unit, activating the pressure-holding telescopic unit to increase pressure, when the internal pressure of the pressure-holding telescopic unit is greater than the internal pressure of the culture kettle, controlling the movable end of the pressure-holding telescopic unit to go down to enter into the culture kettle to grip the handle as the target, then, carrying the handle as the target, by the movable end of the pressure-holding telescopic unit, to return to its original position, disconnecting a connecting channel between the pressure-holding telescopic unit and the culture kettle, relieving pressure by the pressure-holding telescopic unit until the internal pressure of the pressure-holding telescopic unit reaches normal pressure, taking out the handle as the target from the interior of the pressure-holding telescopic unit, and removing the film sheet carried from the handle as the target.
